(19) United States
(12) Patent Application Publication
(10) Pub. No.: US 2019/0284549 A1
(43) Pub. Date: Sep. 19, 2019

(54) METHODS OF DEPLETING OR ISOLATING TARGET RNA FROM A NUCLEIC ACID SAMPLE

(71) Applicant: SMOBiO Technology, Inc., Hsinchu City (TW)

(72) Inventors: KUAN-LIN LEE, Hsinchu City (TW); JIN-LING CHANG, Hsinchu City (TW)

(73) Assignee: SMOBiO Technology, Inc., Hsinchu City (TW)

(21) Appl. No.: 15/920,421

(22) Filed: Mar. 13, 2018

(51) Int. Cl. C12N 15/10; C12Q 1/6811; C40B 40/06; C12N 9/22

(52) U.S. Cl. CPC C12N 15/1013; C12N 15/1096; C12Q 1/6811; C12Y 301/21001; C12N 9/22; C12Y 301/26004; C40B 40/06

(57) ABSTRACT

The present invention relates to methods of depleting or isolating target RNA from a nucleic acid sample.

This application contains a Sequence Listing in computer readable form. The computer readable form is incorporated herein by reference.

### BACKGROUND OF THE INVENTION

### Field of the Invention

The present invention relates to methods of depleting or isolating target RNA from a nucleic acid sample.

### Description of Prior Art

Over the last years, there has been a fundamental shift away from the use of the Sanger method for DNA sequencing to so-called “next generation sequencing” (NGS) technologies. NGS technology requires the preparation of a sequencing library which is suitable for massive parallel sequencing. The sequencing library can be prepared from fragments of genomic DNA or cDNAs which is reverse transcribed from RNA. Generally, among total RNA in the sample, rRNA and tRNA are not target of interest.

Since rRNA comprises over 70% of the total RNA, its presence can complicate various types of analyses of other RNA molecules of interest in a sample (e.g., gene expression analyses by arrays or microarrays, next-generation sequencing of tagged cDNA molecules made from one or more types of RNA molecules in samples (e.g., using the massively parallel digital sequencing methods referred to as “RNA-seq”), etc.). The problems caused by rRNA are especially difficult for analyses of RNA molecules of interest that are fragmented. For example, a considerable and continuing problem in the art is to find better methods for removing degraded rRNA from formalin-fixed paraffin-embedded (FFPE) tissue sections. If better methods were available to remove degraded rRNA from samples (e.g., FFPE-derived samples), it is believed that the enormous quantities of clinical specimens, for which medical outcomes of various diseases and various treatments are recorded in the medical records, would provide extremely valuable information related to identifying RNAs involved in the cause, maintenance, response, diagnosis, or prognosis of many diseases, such as cancer.

EP2464729 discloses methods, compositions, and kits for generating rRNA-depleted samples and for isolating rRNA from samples. In particular, the present invention provides compositions comprising affinity-tagged antisense rRNA molecules corresponding to substantially all of at least one rRNA molecule (e.g., 28S, 26S, 25S, 18S, 5.8S and 5S eukaryotic cytoplasmic rRNA molecules, 12S and 16S eukaryotic mitochondrial rRNA molecules, and 23S, 16S and 5S prokaryotic rRNA molecules) and methods for using such compositions to generate rRNA-depleted samples or to isolate rRNA molecules from samples. The method uses streptavidin as binding matrix to remove biotin-tagged rRNA molecules. However, the preparation of probes (cloning, in vitro transcription, and remove of DNA template) is time-consuming and costly. The kit and composition comprising RNA molecules have to be stored and transported at −70° C.

U.S. Pat. No. 9,005,891 discloses methods of depleting RNA from a nucleic acid sample. The method is useful for depleting RNA from a nucleic acid sample obtained from a fixed paraffin-embedded tissue (FPET) sample. The method may also be used to prepare cDNA, in particular, a cDNA library for further analysis or manipulation. The method uses single strand DNA (ssDNA) probe to hybridize to target RNA and the resulting DNA-RNA hybrid is degraded with RNase. However, only the completely matched ssDNA probe-target RNA hybrid will be degraded by RNase. Only the perfectly matched probe-target RNA hybrid will be degraded by RNase, the probe in the method must be species-specific probe. That is, the method needs to design different probes for different species.

Still further, better methods for removing rRNA, including degraded rRNA, from non-rRNA RNA molecules of interest would greatly improve the applicability and success of methods.

### SUMMARY OF THE INVENTION

The invention provides a method of depleting target RNA from a nucleic acid sample comprising target and non-target RNA molecules, comprising: (a) contacting the nucleic acid sample with at least one double strand DNA probe in a mixture, wherein each strand of the at least one double strand DNA probe is complementary to part of the target RNA and capable of specifically hybridizing to entire full length sequence of the target RNA; and (b) contacting the mixture with RNase that specifically recognizes the DNA-RNA hybrid and degrades the target RNA in the DNA-RNA hybrid.

The invention also provides a method of depleting or isolating target RNA from a nucleic acid sample comprising target and non-target RNA molecules, comprising: (a) contacting the nucleic acid sample with at least one modified double strand DNA probe in a mixture, wherein the at least one modified double strand DNA probe is having at least one nucleotide modified with affinitive moiety or reactive moiety, wherein each strand of the at least one double strand DNA probe is complementary to part of the target RNA and capable of specifically hybridizing to the target RNA; and (b) contacting the mixture with a matrix that specifically interacts with the at least one modified double strand DNA probe on the modified DNA-RNA hybrid, such that the modified DNA-RNA hybrid bind to the matrix and are removed from the mixture, wherein the at least one modified double strand DNA probe is having at least one nucleotide modified with affinitive moiety and the matrix is affinitive matrix, or the at least one modified double strand DNA probe is having at least one nucleotide modified with reactive moiety and the matrix is reactive matrix.

The invention further provides a method of preparing a denatured double strand DNA in a nucleic acid sample for hybridization, comprising: (a) contacting the nucleic acid sample for hybridization with double strand DNA in a hybridization buffer; and (b) heating the mixture to a temperature from 68 to 90° C. to obtain the denatured double strand DNA, wherein the hybridization buffer comprises formamide in a concentration from 40% to 70% by volume.

### BRIEF DESCRIPTION OF THE DRAWINGS

FIG. 1 shows that 16S, 23S rRNA of JM109 total RNA were subtracted by reverse transcribing the rRNA followed by RNase H/DNase I treatment. -: no primers; 16S: primers for subtracting 16S rRNA; 23S: primers for subtracting 23S rRNA.

FIG. 2 shows that 16S, 23S rRNA of JM109 total RNA were subtracted by reverse transcribing the rRNA with biotinylated dNTPs followed by streptavidin-resin capturing. -: no primers; 16S: primers for subtracting 16S rRNA; 23S: primers for subtracting 23S rRNA. (FIG. 2A) Removing Biotin-DNA/RNA hybrid by 20 μl streptavidin-resins is not sufficient. (FIG. 2B) The RNA samples were treated with extra 20 μl resins to eliminate residual DNA hybridized rRNA.

FIG. 3 shows that 16S, 23S rRNA of JM109 total RNA were subtracted by dsDNA probe hybridization followed by RNase H/DNase I treatment. -: no probes; 16S: probes for subtracting 16S rRNA; 23S: probes for subtracting 23S rRNA.

FIG. 4 shows that 16S, 23S rRNA of JM109 total RNA were subtracted by hybridization with biotinylated dsDNA probes followed by streptavidin coated magnetic beads capturing. -: no probes; 16S: probes for subtracting 16S rRNA; 23S: probes for subtracting 23S rRNA. (FIG. 4A) Removing Biotin-DNA/RNA hybrid by 50 μl streptavidin coated magnetic beads is not sufficient. (FIG. 4B) The RNA samples were treated with extra 25 μl beads to eliminate residual DNA hybridized rRNA.

FIG. 5 shows that 18S, 28S rRNA of 293 total RNA were subtracted by hybridization with biotinylated ssDNA probes followed by streptavidin coated magnetic beads capturing. -: no probes; 18S: probes for subtracting 18S rRNA; 28S: probes for subtracting 28S rRNA.

### DETAILED DESCRIPTION OF THE INVENTION

The invention provides a method of depleting target RNA from a nucleic acid sample comprising target and non-target RNA molecules, comprising: (a) contacting the nucleic acid sample with a multiplicity of modified single strand DNA probes in a mixture, wherein the multiplicity of modified single strand DNA probes are complementary to part of the target RNA and capable of specifically hybridizing to 3 to 100% of entire full length sequence of the target RNA, wherein the multiplicity of single strand DNA probes are ranging from 40 to 120 bases; (b) contacting the mixture with a matrix that specifically interacts with the multiplicity of modified single strand DNA probes on a modified DNA-RNA hybrid, such that the modified DNA-RNA hybrid bind to the matrix and are removed from the mixture, wherein the multiplicity of modified single strand DNA probes are having at least one nucleotide modified with affinitive moiety and the matrix is affinitive matrix, or the multiplicity of modified single strand DNA probes are having at least one nucleotide modified with reactive moiety and the matrix is reactive matrix.

The invention provides a method of depleting target RNA from a nucleic acid sample comprising target and non-target RNA molecules, comprising: (a) contacting the nucleic acid sample with a multiplicity of modified single strand DNA probes in a mixture, wherein the multiplicity of modified single strand DNA probes are complementary to part of the target RNA and capable of specifically hybridizing to 3 to 100% of entire full length sequence of the target RNA, wherein the multiplicity of single strand DNA probes are ranging from 40 to 120 bases; (b) contacting the mixture with a matrix that specifically interacts with the multiplicity of modified single strand DNA probes on a modified DNA-RNA hybrid, such that the modified DNA-RNA hybrid bind to the matrix and are removed from the mixture, wherein the multiplicity of modified single strand DNA probes are having affinitive moiety at a ratio of at least one affinitive moiety per every 10 nucleotides and the matrix is affinitive matrix, or the multiplicity of modified single strand DNA probes are having reactive moiety at a ratio of at least one reactive moiety per every 10 nucleotides and the matrix is reactive matrix.

In one embodiment, the multiplicity of modified single strand DNA probes are biotinylated single strand DNA probes and the affinitive matrix is avidin matrix or streptavidin matrix.

In another embodiment, the biotinylated single strand DNA probes are prepared from reacting the multiplicity of modified single strand DNA probes are having at least one nucleotide modified with a first reactive moiety with a biotin modified with a second reactive moiety.

In further embodiment, the first reactive moiety is primary amine group and the second reactive moiety is N-hydroxysuccinimide group.

In another embodiment, the affinitive matrix is prepared from reacting a streptavidin which is modified with a first reactive moiety with a matrix having a second reactive moiety.

In further embodiment, the first reactive moiety is primary amine group and the second reactive moiety is N-hydroxysuccinimide group.

In one embodiment, the reactive moiety is alkyne group and the reactive matrix is containing azide group, the reactive moiety is azide group and the reactive matrix is containing alkyne group, the reactive moiety is thioester group and the reactive matrix is containing N-terminal cysteine group, the reactive moiety is N-terminal cysteine group and the reactive matrix is containing thioester group, the reactive moiety is primary amine group and the reactive matrix is containing N-hydroxysuccinimide group, or the reactive moiety is N-hydroxysuccinimide group and the reactive matrix is containing primary amine group.

In another embodiment, the target RNA is ribosomal RNA or transfer RNA.

In another embodiment, the nucleic acid sample comprise RNA extracted, isolated, or purified from a source selected from the group consisting of: a tissue sample, a cell sample, a paraffin-embedded sample, a paraffin-embedded formalin-fixed (FFPE) sample, and an environmental sample consisting of soil, water, growth medium, or a biological fluid or specimen.

In another embodiment, the matrix is selected from the group consisting of microtitre plate, magnetic bead, non-magnetic bead, sedimentation particle, and affinity chromatography column.

In another embodiment, the multiplicity of modified single strand DNA probes are capable of specifically hybridizing to 25 to 100% of entire full length sequence of the target RNA.

In another embodiment, the multiplicity of modified single strand DNA probes are capable of specifically hybridizing to 75 to 100% of entire full length sequence of the target RNA.

In another embodiment, the multiplicity of modified single strand DNA probes are capable of specifically hybridizing to 100% of entire full length sequence of the target RNA.

In yet another embodiment, the multiplicity of modified single strand DNA probes are having affinitive moiety or reactive moiety at a ratio of at least one affinitive moiety or reactive moiety per every 10 nucleotides of the multiplicity of modified single strand DNA probes.

The invention also provides a method of depleting target RNA from a nucleic acid sample comprising target and non-target RNA molecules, comprising: a) contacting the nucleic acid sample with reverse transcriptase, dNTPs, and at least one DNA primer complementary to part of the target RNA, and reverse transcribing the target RNA to form a DNA-RNA hybrid, thereby generating a treated sample, wherein the at least one DNA primer specifically hybridizes to the target RNA; and (b) contacting the treated sample with RNase that specifically recognizes the DNA-RNA hybrid and degrades the target RNA in the DNA-RNA hybrid. In one embodiment, the method further comprises contacting the treated sample with DNase to degrade residual DNA from the DNA-RNA hybrid after step (b). In another embodiment, the target RNA is ribosomal RNA or transfer RNA.

In one embodiment, the nucleic acid sample comprise RNA extracted, isolated, or purified from a source selected from the group consisting of: a tissue sample, a cell sample, a paraffin-embedded sample, a paraffin-embedded formalin-fixed (FFPE) sample, and an environmental sample consisting of soil, water, growth medium, or a biological fluid or specimen.

In another embodiment, the at least one DNA primer is a segment of DNA complementary to a target RNA sequence and that serve as starting point for DNA synthesis. In further embodiment, the RNase is RNase H. In further embodiment, the DNase is DNase I.

The invention further provides a method of depleting or isolating target RNA from a nucleic acid sample comprising target and non-target RNA molecules, comprising: (a) contacting the nucleic acid sample with reverse transcriptase, dNTPs, at least one modified dNTP, and at least one DNA primer complementary to part of the target RNA, and reverse transcribing the target RNA to form a modified DNA-RNA hybrid, thereby generating a treated sample, wherein the at least one DNA primer specifically hybridizes to the target RNA, the at least one modified dNTP is dNTP with affinitive moiety or dNTP with reactive moiety; and (b) contacting the treated sample with a matrix that specifically interacts with the modified dNTPs on the modified DNA-RNA hybrid, such that the modified DNA-RNA hybrid bind to the matrix and are removed from the treated sample, wherein the modified dNTPs are dNTPs with affinitive moiety and the matrix is affinitive matrix, or the modified dNTPs are dNTPs with reactive moiety and the matrix is reactive matrix.

In one embodiment, the dNTPs with affinitive moiety is biotinylated dNTPs and the affinitive matrix is avidin matrix or streptavidin matrix.

In another embodiment, the biotinylated single strand DNA probes are prepared from reacting the multiplicity of modified single strand DNA probes are having at least one nucleotide modified with a first reactive moiety with a biotin modified with a second reactive moiety.

In further embodiment, the first reactive moiety is primary amine group and the second reactive moiety is N-hydroxysuccinimide group.

In another embodiment, the affinitive matrix is prepared from reacting a streptavidin which is modified with a first reactive moiety with a matrix having a second reactive moiety.

In further embodiment, the first reactive moiety is primary amine group and the second reactive moiety is N-hydroxysuccinimide group.

In another embodiment, the reactive moiety is alkyne group and the reactive matrix is containing azide group, the reactive moiety is azide group and the reactive matrix is containing alkyne group, the reactive moiety is thioester group and the reactive matrix is containing N-terminal cysteine group, the reactive moiety is N-terminal cysteine group and the reactive matrix is containing thioester group, the reactive moiety is primary amine group and the reactive matrix is containing N-hydroxysuccinimide group, or the reactive moiety is N-hydroxysuccinimide group and the reactive matrix is containing primary amine group.

In another embodiment, the target RNA is ribosomal RNA or transfer RNA.

In another embodiment, the nucleic acid sample comprise RNA extracted, isolated, or purified from a source selected from the group consisting of: a tissue sample, a cell sample, a paraffin-embedded sample, a paraffin-embedded formalin-fixed (FFPE) sample, and an environmental sample consisting of soil, water, growth medium, or a biological fluid or specimen.

In another embodiment, the at least one DNA primer is a segment of DNA complementary to a target RNA sequence and that serve as starting point for DNA synthesis.

In further embodiment, the matrix is selected from the group consisting of microtitre plate, magnetic bead, non-magnetic bead, sedimentation particle, and affinity chromatography column.

In the present invention, the advantages of using reverse transcriptase (RTase) include: (a) the process is carried out by design of primer without production of probe; (b) One set of primer design for conserved region can be applied to different similar species due to the product of reverse transcription is perfectly complementary strand.

The invention further provides a method of depleting target RNA from a nucleic acid sample comprising target and non-target RNA molecules, comprising: (a) contacting the nucleic acid sample with at least one double strand DNA probe in a mixture, wherein each strand of the at least one double strand DNA probe is complementary to part of the target RNA and capable of specifically hybridizing to entire full length sequence of the target RNA; and (b) contacting the mixture with RNase that specifically recognizes the DNA-RNA hybrid and degrades the target RNA in the DNA-RNA hybrid. In one embodiment, the method further comprises contacting the mixture with DNase to degrade residual DNA from the DNA-RNA hybrid after step (b).

In another embodiment, the target RNA is ribosomal RNA or transfer RNA.

In another embodiment, the nucleic acid sample comprise RNA extracted, isolated, or purified from a source selected from the group consisting of: a tissue sample, a cell sample, a paraffin-embedded sample, a paraffin-embedded formalin-fixed (FFPE) sample, and an environmental sample consisting of soil, water, growth medium, or a biological fluid or specimen.

In further embodiment, the RNase is RNase H. In further embodiment, the DNase is DNase I.

The invention further provides a method of depleting or isolating target RNA from a nucleic acid sample comprising target and non-target RNA molecules, comprising: (a) contacting the nucleic acid sample with at least one modified double strand DNA probe in a mixture, wherein the at least one modified double strand DNA probe is having at least one nucleotide modified with affinitive moiety or reactive moiety, wherein each strand of the at least one double strand DNA probe is complementary to part of the target RNA and capable of specifically hybridizing to the target RNA; and (b) contacting the mixture with a matrix that specifically interacts with the at least one modified double strand DNA probe on the modified DNA-RNA hybrid, such that the modified DNA-RNA hybrid bind to the matrix and are removed from the mixture, wherein the at least one modified double strand DNA probe is having at least one nucleotide modified with affinitive moiety and the matrix is affinitive matrix, or the at least one modified double strand DNA probe is having at least one nucleotide modified with reactive moiety and the matrix is reactive matrix.

In one embodiment, the at least one modified double strand DNA probe is biotinylated double strand DNA probe and the affinitive matrix is avidin matrix or streptavidin matrix.

In another embodiment, the biotinylated double strand DNA probes are prepared from reacting the multiplicity of modified single strand DNA probes are having at least one nucleotide modified with a first reactive moiety with a biotin modified with a second reactive moiety.

In further embodiment, the first reactive moiety is primary amine group and the second reactive moiety is N-hydroxysuccinimide group.

In another embodiment, the affinitive matrix is prepared from reacting a streptavidin which is modified with a first reactive moiety with a matrix having a second reactive moiety.

In further embodiment, the first reactive moiety is primary amine group and the second reactive moiety is N-hydroxysuccinimide group.

In another embodiment, the reactive moiety is alkyne group and the reactive matrix is containing azide group, the reactive moiety is azide group and the reactive matrix is containing alkyne group, the reactive moiety is thioester group and the reactive matrix is containing N-terminal cysteine group, the reactive moiety is N-terminal cysteine group and the reactive matrix is containing thioester group, the reactive moiety is primary amine group and the reactive matrix is containing N-hydroxysuccinimide group, or the reactive moiety is N-hydroxysuccinimide group and the reactive matrix is containing primary amine group.

In another embodiment, the target RNA is ribosomal RNA or transfer RNA.

In another embodiment, the nucleic acid sample comprise RNA extracted, isolated, or purified from a source selected from the group consisting of: a tissue sample, a cell sample, a paraffin-embedded sample, a paraffin-embedded formalin-fixed (FFPE) sample, and an environmental sample consisting of soil, water, growth medium, or a biological fluid or specimen.

In yet another embodiment, the matrix is selected from the group consisting of microtitre plate, magnetic bead, non-magnetic bead, sedimentation particle, and affinity chromatography column.

The invention further provides a method of preparing a denatured double strand DNA in a nucleic acid sample for hybridization, comprising: (a) contacting the nucleic acid sample for hybridization with double strand DNA in a hybridization buffer; and (b) heating the mixture to a temperature from 68 to 90° C. to obtain the denatured double strand DNA, wherein the hybridization buffer comprises formamide in a concentration from 40% to 70% by volume.

In general condition, the double strand DNA is denatured at temperatures greater than 90° C. However, RNA is more prone to hydrolysis at such high temperature. In the method of the present invention, there is no need to denature double strand DNA at temperatures greater than 90° C. Therefore, the probability of RNA hydrolysis is decreased in the present invention.

In another embodiment, the temperature is 70° C. In another embodiment, the formamide is in a concentration of 40% by volume.

Nucleic acids such as DNA and/or RNA can be isolated from a sample of interest according to methods known in the prior art to provide the starting material for preparing the sequencing library. RNA is usually first transcribed into cDNA prior to preparing the sequencing library. The term “sample” is used herein in a broad sense and is intended to include a variety of sources and compositions that contain nucleic acids. The sample may be a biological sample but the term also includes other, e.g. artificial samples which comprise nucleic acids such as e.g. PCR products or compositions comprising already purified nucleic acids. Exemplary samples include, but are not limited to, whole blood; blood products; red blood cells; white blood cells; buffy coat; swabs; urine; sputum; saliva; semen; lymphatic fluid; amniotic fluid; cerebrospinal fluid; peritoneal effusions; pleural effusions; biopsy samples; fluid from cysts; synovial fluid; vitreous humor; aqueous humor; bursa fluid; eye washes; eye aspirates; plasma; serum; pulmonary lavage; lung aspirates; animal, including human or plant tissues, including but not limited to, liver, spleen, kidney, lung, intestine, brain, heart, muscle, pancreas, cell cultures, as well as lysates, extracts, or materials and fractions obtained from the samples described above or any cells and microorganisms and viruses that may be present on or in a sample and the like. Materials obtained from clinical or forensic settings that contain nucleic acids are also within the intended meaning of the term “sample”. Preferably, the sample is a biological sample derived from a human, animal, plant, bacteria or fungi. Preferably, the sample is selected from the group consisting of cells, tissue, tumor cells, bacteria, virus and body fluids such as for example blood, blood products such as buffy coat, plasma and serum, urine, liquor, sputum, stool, CSF and sperm, epithelial swabs, biopsies, bone marrow samples and tissue samples, preferably organ tissue samples such as lung, kidney or liver. The term “sample” also includes processed samples such as preserved, fixed and/or stabilized samples.

As used herein, the term “double strand DNA probe” refers to a DNA oligonucleotide having a sequence partly or completely complementary to a “target RNA” and specifically hybridizes to the RNA. As used herein, “target RNA” refers to an undesired RNA that is the target for depletion from the nucleic acid sample. The target RNA may be any RNA, including, but not limited to, rRNA, tRNA, and mRNA. DNA probes may be produced by techniques known in the art such as chemical synthesis and by in vitro or in vivo expression from recombinant nucleic acid molecules. The DNA probes may also be produced by amplification of the target RNA, including, but not limited to, RT-PCR. In one embodiment of the invention, a single DNA probe spans the entire length of the target RNA. DNA probes may or may not have regions that are not complementary to a target RNA, so long as such sequences do not substantially affect specific hybridization to the RNA. In another embodiment of the invention, the DNA probe may be complementary to all or part of a target RNA sequence and therefore, there may be more than one DNA probe that specifically hybridizes to the RNA. For example, there may be at least 1, 2, 3, 4, 5, 6, 7, 8, 9, or 10 DNA probes that specifically hybridize to a RNA. The DNA probes may be complementary to sequences that overlap one another, or may be complementary to non-overlapping sequences.

As used herein, “specifically hybridizes” refers to a state where a specific DNA probe is able to hybridize with a target RNA, for example, rRNA, over other nucleic acids present in a nucleic acid sample. The DNA probe is first denatured into single-stranded DNA by methods known in the art, for example, by heating or under alkaline conditions, and then hybridized to the target RNA by methods also known in the art, for example, by cooling the heated DNA in the presence of the target RNA. The condition under which a DNA probe specifically hybridizes with an RNA are well known to those of ordinary skill in the art and it will be appreciated that these conditions may vary depending upon factors including the GC content and length of the probe, the hybridization temperature, the composition of the hybridization reagent or solution, and the degree of hybridization specificity sought.

As used herein, the term “complementary” refers to a nucleic acid comprising a sequence of consecutive nucleobases capable of hybridizing to another nucleic acid strand even if less than all the nucleobases do not base pair with a counterpart nucleobase. In certain embodiments, a “complementary” nucleic acid comprises a sequence of the nucleobase sequence is capable of base-pairing with another nucleic acid sequence through hybridization carried out by heating and then cooling to room temperature to form stable structure of probe and target RNA.

In the present invention, the advantage of using double strand DNA (dsDNA) probe is that preparation of probe (such as PCR) is easy and inexpensive.

### EXAMPLES

The examples below are non-limiting and are merely representative of various aspects and features of the present invention.

In one embodiment, the total RNA used herein was extracted from E coli JM109 strain using 3-zol reagent (MDbio, Inc, Taiwan)

### Example 1

rRNA was reverse transcribed with target primer mixture and reverse transcriptase (RTase). The rRNA that hybridized with complementary DNA was then digested with RNase H. The rest DNA was then digested DNase I.

(1) 10 μg JM109 RNA was mixed with 0.5 μl primer mixture (each 12.5 μM) (Table. 1) in 25 μA To hybridize primers, the mixture was heated to 70° C. for 5 min and then soon on ice for 1 min.

TABLE 1
Primers for reverse transcription

| | | SEQ |
|---|---|---|
| | | ID |
| For depleting 16S rRNA, mix r16S-1~4 primers | | NO |
| r16S-1 | CAGTAAGGAGGTGATCCAACCGCAGGTT | 109 |
| r16S-2 | CCAACATTTCACAACACGAGCTGACGACAG | 110 |
| r16S-3 | CTCTACGCATTTCACCGCTACACCTGG | 111 |
| r16S-4 | CCCGTAGGAGTCTGGACCGTGTCTCAGTT | 112 |
| | | SEQ |
| | | ID |
| For depleting 23S rRNA, mix r23S-1~8 primers | | NO |
| r23S-1 | CAGAAGGTTAAGCCTCACGGTTCATTAGT | 113 |
| r23S-2 | CCCAGGATGTGATGAGCCGACATCGAGGT | 114 |
| r23S-3 | CCATGCAGACTGGCGTCCACACTTCAAAG | 115 |
| r23S-4 | CCACTTTCGTGTTTGCACAGTGCTGTGTTT | 116 |
| r23S-5 | CCTTCGCAGTAACACCAAGTACAGGAATAT | 117 |
| r23S-6 | CCCACATCGTTTCCCACTTAACCATGACTT | 118 |
| r23S-7 | CCCAGTTAAGACTCGGTTTCCCTTCGGCT | 119 |
| r23S-8 | CCCTGTATCGCACGCCTTTCCAGACGCTT | 120 |

(2) 25 μl reverse transcription mixture containing 2×RT buffer, RTase (SMOBIO), RNase Inhibitor (RI) (SMOBIO), dNTPs (SMOBIO) were added to the mixture of (1) and then placed on 37° C. for 5 min.

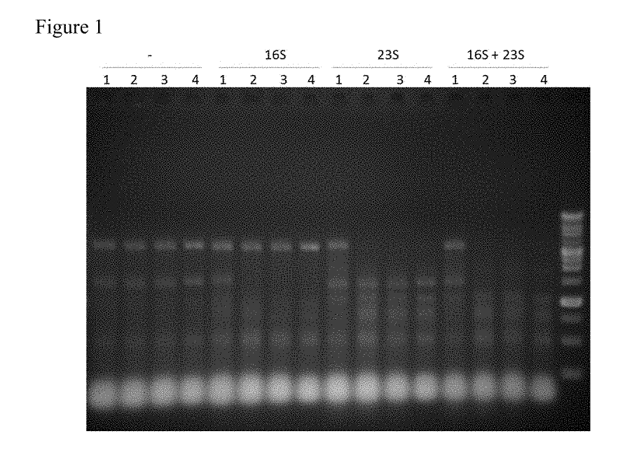

(3) 5 μl of the mixture of (2) was kept for gel loading (FIG. 1; Label 1). 1 μl RNase H (NEB) was added to residual 45 μl reaction mixtures and kept at 37° C. for 30 min.

(4) 5 μl of (3) was kept for gel loading (FIG. 1; Label 2). The rest reaction mixtures were separated to two tubes for DNase I digestion. 10 μl was taken and 1 μl DNase I (Roche) was added and placed on 37° C. for 30 min. The result is showed in FIG. 1, Label 3. The rest 30 μl was diluted to 150 μl while adding 3 μl DNase I and final in 1× DNase I reaction buffer. After at 37° C. for 30 min, the mixture was cleaned up by RNA PURE Kit (Geneaid) and resolved in 30 μl volume. The result is showed in FIG. 1, Label 4. Two groups of DNase I treatments showed no difference.

### Example 2

rRNA was reverse transcribed with target primer mixture, 50% biotinylated dCTP and reverse transcriptase (RTase). The rRNA that hybridized with complementary and biotinylated DNA was then removed by streptavidin-resins.

(1) 10 μg JM109 RNA was mixed with 0.5 μl primer mixture (each 12.5 μM) (Table. 1) in 25 μl. To hybridize primers, the mixture was heated to 70° C. for 5 min and then soon on ice for 1 min.

(2) 25 μl reverse transcription mixture containing 2×RT buffer, RTase (SMOBIO), RI (SMOBIO), dNTPs (SMOBIO) were added to (1), wherein the dNTPs contain 50% biotinylated dCTP (Roche). Then placed on 50° C. for 15 min.

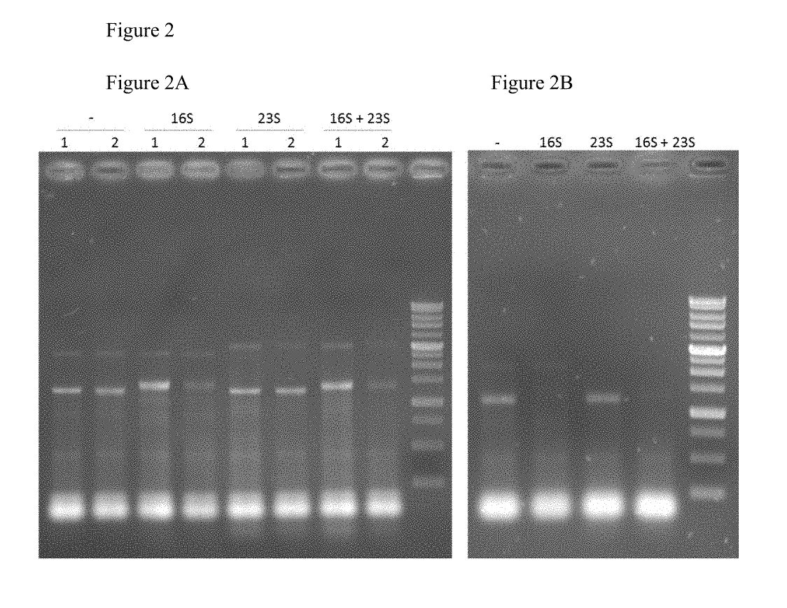

(3) The mixture was cleaned up by RNA PURE Kit (Geneaid) to eliminate excess biotinylated dCTP. 5 μl was kept for gel loading (FIG. 2A; Label 1).

(4) Biotin-DNA/RNA hybrid was removed by streptavidin-resins (PIRECE). 20 μl streptavidin-resins was washed twice with DEPC-treated ddH₂O and once with 1× binding buffer (5 mM Tris-HCl pH7.5, 0.5 mM EDTA, 1M NaCl, 0.05% Tween20). Then 40 μl 2× binding buffer, RI (SMOBIO), 30 μl elution product of (3) was added. Keep swirling at room temperature for 30 min and then 50° C. for 5 min.

(5) The mixture was cleaned up by RNA PURE Kit (Geneaid). 5 μl was kept for gel loading (FIG. 2A; Label 2).

(6) The result showed that 20 μl streptavidin-resins was not enough to subtract targeted rRNA entirely. Another 20 μl streptavidin-resins was used to subtract all targeted rRNA. The procedure was the same as step (4) to (5). The result was showed in FIG. 2B.

### Example 3

rRNA was hybridized with dsDNA probes. The rRNA that hybridized with complementary DNA was then digested with RNase H. The rest DNA was then digested DNase I. dsDNA probes hybridization→RNase H→DNase I

(1) dsDNA probes preparation. dsDNA probes were prepared by PCR using Taq DNA polymerase (SMOBIO), dNTPs (SMOBIO), E. coli W3110 gDNA as template, the primers were listed in Table 2. The 16S probes were made by mixing 16S-1˜4 PCR products in the same molar ratio to a final concentration 400 ng/μL. The 23S probes were made by mixing 23S-1˜8 PCR products in the same molar ratio to a final concentration 400 ng/μL.

TABLE 2
Primers for producing probes.

| paired primers for producing 16S probes | | SEQ ID NO |
|---|---|---|
| 16S-1F | CAGTAAGGAGGTGATCCAACCGCAGGTT | 121 |
| 16S-1R | GTTAAGTCCCGCAACGAGCGCA | 122 |
| 16S-2F | CCAACATTTCACAACACGAGCTGACGACAG | 123 |
| 16S-2R | ATCTGGAGGAATACCGGTGGCG | 124 |
| 16S-3F | CTCTACGCATTTCACCGCTACACCTGG | 125 |
| 16S-3R | AGGCAGCAGTGGGGAATATTGCA | 126 |
| 16S-4F | CCCGTAGGAGTCTGGACCGTGTCTCAGTT | 127 |
| 16S-4R | GCGGATCCAAATTGAAGAGTTTGATCATGG | 128 |
| paired primers for producing 23S probes | | SEQ ID NO |
| 23S-1F | CAGAAGGTTAAGCCTCACGGTTCATTAGT | 129 |
| 23S-1R | GCTGAAGTAGGTCCCAAGGGTA | 130 |
| 23S-2F | CCCAGGATGTGATGAGCCGACATCGAGGT | 131 |
| 23S-2R | AGCCGACCTTGAAATACCACCC | 132 |
| 23S-3F | CCATGCAGACTGGCGTCCACACTTCAAAG | 133 |
| 23S-3R | ACGTATACGGTGTGACGCCTGC | 134 |
| 23S-4F | CCACTTTCGTGTTTGCACAGTGCTGTGTTT | 135 |
| 23S-4R | GGGGACGGAGAAGGCTATGTTG | 136 |
| 23S-5F | CCTTCGCAGTAACACCAAGTACAGGAATAT | 137 |
| 23S-5R | AAGGCCCAGACAGCCAGGATGT | 138 |
| 23S-6F | CCCACATCGTTTCCCACTTAACCATGACTT | 139 |
| 23S-6R | CGTTAAGTTGCAGGGTATAGAC | 140 |
| 23S-7F | CCCAGTTAAGACTCGGTTTCCCTTCGGCT | 141 |
| 23S-7R | TGACAGCCCCGTACACAAAAAT | 142 |
| 23S-8F | CCCTGTATCGCACGCCTTTCCAGACGCTT | 143 |
| 23S-8R | AAGGATCCGGTTAAGCGACTAAGCGTACAC | 144 |

(2) Targeted rRNA was mixed with 2× probes by weight. 1 μg JM109 RNA was mixed with 400 ng 16S, 1.1 μg 23S, or 400 ng 16S+1.1 μg 23S biotinylated dsDNA probe mixture in 40 μl in a final concentration of 50 mM Tris-HCl, pH7.5, 100 mM NaCl, and 40% formamide. To hybridize probes, the mixture was heated to 70° C. for 5 min and then slowly cooled down to 25° C. In one embodiment, the procedure was done in a thermal cycler and the program was set as follow:

| | |
|---|---|
| 70° C. | 5 min |
| 65° C. | 1 min |
| 60° C. | 1 min |
| 55° C. | 1 min |
| 50° C. | 1 min |
| 25° C. | 1 min |

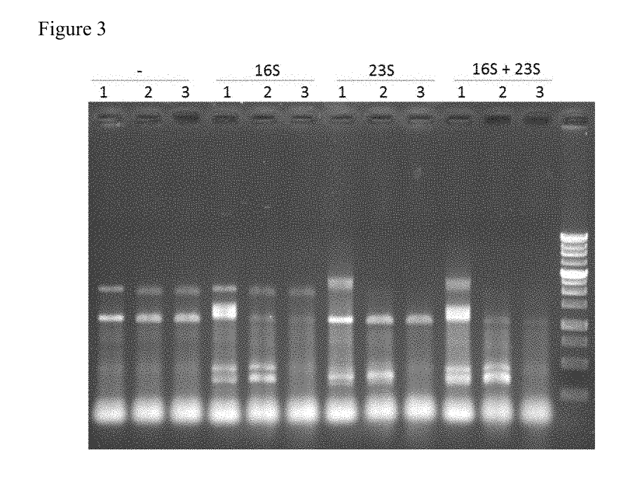

(3) After probe hybridization, the mixture was cleaned up by RNA PURE Kit (Geneaid) and resolved in 40 μl volume. 5 l was kept for gel loading (FIG. 3; Label 1). 1 μl RNase H (NEB) and 4 μl 10× RNase H buffer were added to the residual 350, and then kept at 37° C. for 30 min.

(4) 5.7 μl of (3) was kept for gel loading (FIG. 3; Label 2). 1 μl DNase I (Roche) was added to the rest reaction mixtures and placed on 37° C. for 30 min. The result was showed in FIG. 3, Label 3.

### Example 4

rRNA was hybridized with biotinylated DNA probes. The rRNA that hybridized with complementary and biotinylated DNA was then removed by streptavidin-resins.

(1) Biotinylated dsDNA probes preparation. Biotinylated dsDNA probes were prepared as mentioned above except that dNTPs used here containing 50% biotinylated dCTP (Roche). The 16S probes were made by mixing 16S-1˜4 PCR products in the same molar ratio to a final concentration 400 ng/4. The 23S probes were made by mixing 23S-1˜8 PCR products in the same molar ratio to a final concentration 400 ng/A.

(2) Targeted rRNA was mixed with 2× probes by weight. 1 μg JM109 RNA was mixed with 400 ng 16S, 1.1 μg 23S, or 400 ng 16S+1.1 μg 23S biotinylated dsDNA probe mixture in 40 μl in a final concentration of 50 mM Tris-HCl, pH7.5, 100 mM NaCl, and 40% formamide. To hybridize probes, the mixture was heated to 70° C. for 5 min and then slowly cooled down to 25° C. In one embodiment, the procedure was done in a thermal cycler and the program was set as follow:

| | |
|---|---|
| 70° C. | 5 min |
| 65° C. | 1 min |
| 60° C. | 1 min |
| 55° C. | 1 min |
| 50° C. | 1 min |
| 25° C. | 1 min |

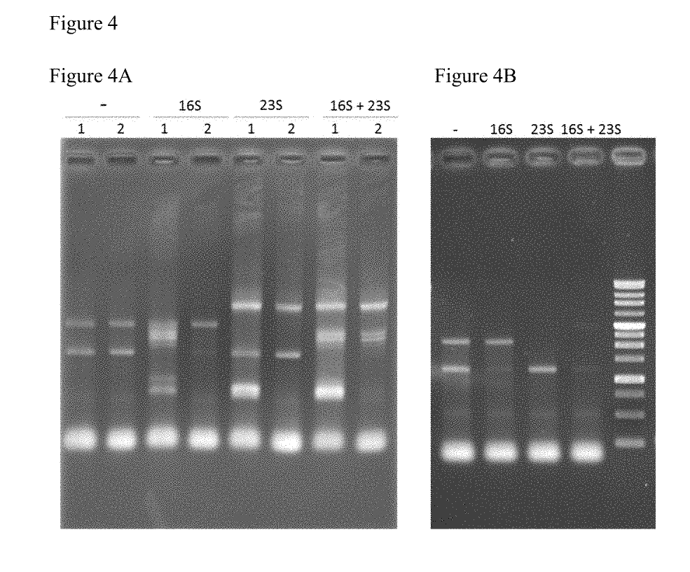

(3) After probe hybridization, the mixture was cleaned up by RNA PURE Kit (Geneaid). 5 μl was kept for gel loading (FIG. 4A; Label 1).

(4) Biotin-DNA/RNA hybrid was removed by streptavidin coated magnetic beads (SMOBIO). 50 μl streptavidin coated magnetic beads was washed twice with DEPC-treated ddH₂O and once with 1× binding buffer (5 mM Tris-HCl pH7.5, 0.5 mM EDTA, 1M NaCl, 0.05% Tween20). Then 40 μl 2× binding buffer, RI(SMOBIO), 30 μl elution product of (3) were added. Keep swirling at room temperature for 30 min and then 50° C. for 5 min.

(5) The mixture was cleaned up by RNA PURE Kit (Geneaid), and 5 μl was kept for gel loading (FIG. 4A; Label 2).

(6) The result showed that 50 μl streptavidin coated magnetic beads was not sufficient to subtract targeted rRNA entirely. Another 25 μl streptavidin coated magnetic beads was added to subtract residual targeted rRNA. The procedure was the same as steps (4)-(5). The result was showed in FIG. 4B.

### Example 5: Probes Preparation

Sequences of the modified single strand DNA probes targeting the full length sequences of human 18S and human 28S rRNA are shown in Table 1. Wherein A means dA, T means dT, C means dC, G means dG, and I means amino-dT. DNA probes were synthesized by ABI DNA synthesizer with regular DMT-dN phosphoramidites and Amino-Modifier-C6-dT-CE phosphoramidite (Link Technologies Ltd., Scotland). After synthesis, modified DNA probes were treated with Sulfo-NHS-Biotin (ApexBio technology LLC, Houston, USA) for biotin labeling. In detail, DNA oligonucleotides were resolved in 0.1 M sodium bicarbonate to 200 μM. Sulfo-NHS-biotin was dissolved in 0.1 M sodium bicarbonate to 16 mM. Mix equal volume of probes and Sulfo-NHS-biotin solution and stay at room temperature overnight for labeling reaction. And then use desalt column to remove extra biotin. DNA probes can also be directly synthesized by ABI DNA synthesizer with regular DMT-dN phosphoramidites and biotin-dT-CE Phosphoramidite. After synthesis, DNA probes would be biotin labeled probes. No further reaction as above mentioned is required.

TABLE 3
modified single strand DNA probes

| | Sequences | SEQ ID NO |
|---|---|---|
| 18S-1 | IAATGATCCITCCGCAGGITCACCIACGGAAACCITGTTACGACITTTACTTCCICTAGAIAGT | 1 |
| 18S-2 | AAGITCGACCGICITCICAGCGCICCGCCAGGGCCGIGGGCCGACCCIGGCGGGGCCGAICCGA | 2 |
| 18S-3 | GGCCICACIAAACCAICCAAICGGTAGIAGCGACGGGCGGIGTGIACAAAGGICAGGGACITAA | 3 |
| 18S-4 | ICAACGCAAGCITATGACCCGCACITACIGGGAAITCCTCGITCATGGGGAAIAATTGCAAICC | 4 |
| 18S-5 | CGAICCCCAICACGAAIGGGGITCAACGGGITACCCGCGCCIGCCGGCGIAGGGIAGGCACACG | 5 |
| 18S-6 | IGAGCCAGICAGTGIAGCGCGCGIGCAGCCCCGGACAICTAAGGGCAICACAGACCIGTTATIG | 6 |
| 18S-7 | ICAATCICGGGIGGCIGAACGCCACTTGICCCTCIAAGAAGTIGGGGGACGCCGACCGCICGGG | 7 |
| 18S-8 | GICGCGTAACTAGITAGCAIGCCAGAGICTCGTTCGTIATCGGAATIAACCAGACAAAICGCIC | 8 |
| 18S-9 | ACCAACIAAGAICGGCCAIGCACCACCAICCACGGAAICGAGAAAGAGCIATCAAICTGICAAT | 9 |
| 18S-10 | CTGICCGTGICCGGGCCGGGIGAGGTTICCCGTGITGAGTCAAATIAAGCCGCAGGCICCACIC | 10 |
| 18S-11 | TGGIGGTGCCCTICCGTCAATICCTTTAAGITTCAGCTITGCAACCAIACICCCCCIGGAACCC | 11 |
| 18S-12 | AAGACITTGGTTICCCGGAAGCIGCCCGGCGGGICAIGGGAAIAACGCCGCCGCAICGCCGGIC | 12 |
| 18S-13 | GCAICGTTTAIGGICGGAACIACGACGGIATCTGATCGICTTCGAACCICCGACTTTCGITCTT | 13 |
| 18S-14 | ATIAATGAAAACAITCTIGGCAAAIGCTTTCGCICTGGTCCGTCTIGCGCCGGICCAAGAAITT | 14 |
| 18S-15 | ACCICTAGCGGCGCAAIACGAAIGCCCCCGGCCGICCCTCTIAATCAIGGCCICAGTICCGAAA | 15 |
| 18S-16 | ACCAACAAAAIAGAACCGCGGICCTATICCATTATICCIAGCIGCGGTAICCAGGCGGCICG | 16 |
| 18S-17 | GGCCIGCTTIGAACACICTAATTTITTCAAAGIAAACGCTICGGGCCICGCGGGACACICAGCT | 17 |
| 18S-18 | AAGAGCAICGAGGGGICGCCGAGAGICAAGGGICGGGGACIGGCGGIGGCICGCCICGCGGCGG | 18 |
| 18S-19 | ACCGICCGCCCGCICCCAAGAICCAACIACGAGCITTTTAACIGCAGCAACTTIAATAIACGCT | 19 |
| 18S-20 | ATIGGAGCIGGAATIACCGCGGCTGCIGGCACCAGACITGCCCICCAATGGAICCTCGTIAAAG | 20 |
| 18S-21 | GATTIAAAGTGGACICATTCCAATIACAGGGCCICGAAAGAGICCTGIATTGTTAITTTTCGIC | 21 |
| 18S-22 | ACIACCTCCCCGGGICGGGAGIGGGTAATTIGCGCGCCIGCTGCCITCCTTGGAIGTGGIAGCC | 22 |
| 18S-23 | GTTICTCAGGCICCCTCTCCGGAAICGAACCCIGATTCCCCGICACCCGIGGTCACCAIGGIAG | 23 |
| 18S-24 | GCACGGCGACIACCAICGAAAGITGAIAGGGCAGACGITCGAAIGGGTCGICGCCGCCACIGGG | 24 |
| 18S-25 | GCGIGCGAICGGCCCGAGGITATCIAGAGICACCAAAGCCGICGGCGCCCGICCCCCGGCCIGG | 25 |
| 18S-26 | CCIGAGAGGGGCIGACCGGGITGGTTTIGATCTGAIAAAIGCACGCAICCCCCCCGIGAAGGGG | 26 |
| 18S-27 | ICAGCGCCCGICGGCAIGTATTAGCICTAGAATIACCACAGTTAICCAAGIAGGAGAGGAGIGA | 27 |
| 18S-28 | GCGAICAAAGGAACCAIAACIGATTTAAIGAGCCAITCGCAGITTCACTGIACCGGCCGIGCGT | 28 |
| 18S-29 | ACICAGACAIGCATGGCTIAATCTTIGAGACAAGCAIATGCTACIGGCAGGAICAACCAGGIA | 29 |
| 28S-1 | GACAAACCCITGTGICGAGGGCIGACTTICAAIAGAICGCAGCGAGGGAGCIGCTCTGCIACGT | 30 |
| 28S-2 | ACGIAACCCCIACCCAGIAGCAGGICGTCIACGAAIGGTTIAGCGCCAGGTICCCCACGAACGT | 31 |
| 28S-3 | GCGGIGCGIGACGGGCGAGGIGGCGGCCGCCICICCGGCCGIGCCCCGTTICCCAGGAIGAAGG | 32 |
| 28S-4 | GCACICCGCACCIGACCCCGGICCCGGCGCICGGCGGGGIACGCGCCCICCCGIGCICGCGGGG | 33 |
| 28S-5 | CGCGIGGAGGIGGGGGGCGGCCIGCCGGCGGGIACAGGCGGIGGACCGGCIAICCGAGICCAAC | 34 |
| 28S-6 | GAGGCICCGCGGCGCIGCCGTAICGTICCGCCIGGGCGGGATICTGACTIAGAGGCGTICAGTC | 35 |
| 28S-7 | AIAAICCCACAGATGGIAGCTICGCCCCATIGGCTCCICAGCCAAGCACAIACACCAAAIGTCT | 36 |
| 28S-8 | GAACCIGCGGITCCTCICGTACIGAGCAGGAITACCAIGGCAACAACACAICATCAGIAGGGTA | 37 |
| 28S-9 | AAACIAACCTGTCICACGACGGTCIAAACCCAGCICACGITCCCTATIAGTGGGIGAACAAICC | 38 |
| 28S-10 | ACGCITGGTGAATICTGCTICACAATGAIAGGAAGIGCCGACAICGAAGGAICAAAAIGCGACG | 39 |
| 28S-11 | ICGCTAIGAACGCTIGGCCGCCACAAGCCAGITAICCCTGTGGIAACTTTTCIGACACCICCTG | 40 |
| 28S-12 | CTIAAAACCCAAAAGGICAGAAGGAICGIGAGGCCCCGCITTCACGGICTGIATTCGIACTGAA | 41 |
| 28S-13 | AAICAAGAICAAGCGAGCITTTGCCCITCTGCICCACGGGAGGITTCTGICCTCCCIGAGCTCG | 42 |
| 28S-14 | CCTIAGGACACCIGCGTIACCGTTIGACAGGTGIACCGCCCCAGICAAACICCCCACCIGGCAC | 43 |
| 28S-15 | IGICCCCGGAGCGGGICGCGCCIGGCCGICGCGCGGCCGIGCGCTIGGCGCCAGAAGIGAGAGC | 44 |
| 28S-16 | CCICGGGGCICGCCCCCCCGCCICACCGGGICAGIGAAAAAACGAICAGAGTAGIGGTAITTCA | 45 |
| 28S-17 | CGGCIGCCCGCIGGGICGGCGGACCICGCCICGGGCCCCICGCGGGGACAICGGIGGGGCGCCG | 46 |
| 28S-18 | GGGCCICCCACITATTCIACACCTCICATGTCICTTCACCGIGCCAGACTAGAGICAAGCICAA | 47 |
| 28S-19 | CAGGGICTTCTTICCCCGCIGATICCGCCAAGCCCGITCCCTIGGCTGIGGTTTCGCIGGATAG | 48 |
| 28S-20 | TAGGIAGGGACAGIGGGAAICTCGITCAICCATTCAIGCGCGTCACIAATTAGAIGACGAGGCA | 49 |
| 28S-21 | TTIGGCTACCITAAGAGAGICATAGTTACICCCGCCGTTIACCCGCGCITCATIGAATITCTTC | 50 |
| 28S-22 | ACTTIGACATICAGAGCACIGGGCAGAAAICACAICGCGICAACACCCGCIGCGGGCCITCGCG | 51 |
| 28S-23 | ATGCTITGTTTTAATIAAACAGICGGATICCCCTGGICCGCACCAGITCTAAGICGGCIGCTAG | 52 |
| 28S-24 | CGCCGICCGAGICGAGGIGCCGCGCIGAACCGIGGCCCIGGGGGCGGACCCGICGGIGGGGACC | 53 |
| 28S-25 | CCCGIGGCCCCICCGCCGCCIGCCGCCGICGCCGCCGIGCGCCGIGGAGGAIGGIGGAACGGGG | 54 |
| 28S-26 | GCGIACGGGGICGGGGGGGIAGGGCGGGGGIACGAACCGICCCGCICCGCCGICCGCIGACCGC | 55 |
| 28S-27 | GCCGICCGACCGCICCCCGCCCCIAGCGGACICGCGCGCIACGAGACGIGGGGIGGGGGIGGGG | 56 |
| 28S-28 | GCICGCCGICGCCCGCIGGGCICCCCGGGGGCGICCGCGACGCCIGCCGCAGCIGGGGCGAICC | 57 |
| 28S-29 | ACGGGAAGIGCCCGGCICGCGICCAGAGICGCCGCIGCCGCCGGCICCCCGGGIGCCCIGGCCC | 58 |
| 28S-30 | CCCICGCGIGGGACCGIGCCCCIGCCGCCGGGGCCICGCGGCGGGCIGCIGCCGGCCCCIGCCG | 59 |
| 28S-31 | CCCCIACCCITCICCCCCCGCCGCCGICCCCACGCGGIGCICCCCCGGGGAIGGGGIAGGACGG | 60 |
| 28S-32 | AGCGGIGGAGAGAGAIAGAGAIAGGGCICGGIGCGGGGAGGIAGCGAGCGGCGIGCGCGGGGIG | 61 |
| 28S-33 | GGICGGGGGAGGGICGCGAGIGGGGIGCCCCGGGCGIGGGGGGGGCGICGGCGCCICGICCAGC | 62 |
| 28S-34 | GIGGIGCGCGCCCAICCCCGCTICGCGCCCIAGCCCGACCGAICCAGCCCITAGAGCCAAICCT | 63 |
| 28S-35 | TAICCCGAAGTIACGGATCCGGCITGCCGACITCCCITACCIACATTGTICCAACAIGCCAGAG | 64 |
| 28S-36 | GCIGTTCACCTIGGAGACCIGCIGCGGAIATGGGIACGGCCCGGCGCGAGAITTACACCCICTC | 65 |
| 28S-37 | CCCCGGAITTICAAGGGCIAGCGAGAGCICACCGGAIGCCGCCGGAICCGCGACGCITICCAAG | 66 |
| 28S-38 | GCACGGGCCCCICTCICGGGGIGAACCCATICCAGGGIGCCCIGCCCTICACAAAGIAAAGAGA | 67 |
| 28S-39 | ACTCICCCCGGGGCICCCGCCGGCTICTCCGGGAICGGICGCGITACCGCACIGGACGCCICGC | 68 |
| 28S-40 | GGCGCCCAICICCGCCACICCGGATICGGGGATCIGAACCCGACICCCITTCGAICGGCCGAGG | 69 |
| 28S-41 | CAACGIAGGCCAICGCCCGICCCTICGGAACGGCGCICGCCCAICTCICAGGACCGACIGACCC | 70 |
| 28S-42 | ATGITCAACTGCIGTTCACAIGGAACCCTICTCCACTICGGCCTICAAAGITCTCGTTIGAATA | 71 |
| 28S-43 | TTIGCTACIACCACCAAGAICIGCACCIGCGGCGGCICCACCCGGGCCCGCGCCCIAGGCITCA | 72 |
| 28S-44 | AGGCICACCGCAGCGGCCCICCIACTCGICGCGGCGIAGCGICCGCGGGGCICCGGGIGCGGGG | 73 |
| 28S-45 | AGCIGGGCGIGGGCGGIAGGAGGGIAGGAGGCGIGGGGGGGIGGGCGGGGGAAIGAICCCACAC | 74 |
| 28S-46 | CCCCGICGCCGCCGCIGCCICCGCCCICCGACGIACACCACAIGCGCGCGCICGCICGCCGCCC | 75 |
| 28S-47 | CCGCCGCICCCGICCACTCICGACIGCCGGCGAIGGCCGGGIAIGGGCCCGACGCICCAGCGCC | 76 |
| 28S-48 | AICCATTTICAGGGCTAGITGATICGGCAGGIGAGTTGTIACACACTCCITAGCGGATICCGAC | 77 |
| 28S-49 | TTCCAIGGCCACCGICCTGCTGICTATAICAACCAACACCITTTCIGGGGTCIGATGAGCGICG | 78 |
| 28S-50 | GCAICGGGCGCCTIAACCCGGCGTICGGITCAICCCGCAGCGCCAGITCTGCTIACCAAAAGIG | 79 |
| 28S-51 | GCCCACIAGGCACICGCATICCACGCCCGGCICCACGCCAGIGAGCCGGGCITCTIACCCAITT | 80 |
| 28S-52 | AAAGITTGAGAAIAGGITGAGAICGTTICGGCCCCAAGACCICTAATCATICGCTTIACCGGAT | 81 |
| 28S-53 | AAAACIGCGIGGCGGGGGIGCGICGGGTCIGCGAGAGCGCCAGCIATCCIGAGGGAAACITCGG | 82 |
| 28S-54 | AGGGAACCAGCIACIAGATGGTICGATIAGTCTTICGCCCCTAIACCCAGGICGGAIGACCGAT | 83 |
| 28S-55 | TIGCACGICAGGACCGCIACGGACCICCACCAGAGITTCCICTGGCITCGCCCIGCCCAGGCAT | 84 |
| 28S-56 | AGTICACCATCTTICGGGTCCIAACACGIGCGCICGTGCICCACCICCCCGGCGCGGCIGGCGA | 85 |
| 28S-57 | GACGGGCCGGIGGIGCGCCCICGGCGGACIGGAGAGGCCICGGGAICCCACCICGGCCIGCGAG | 86 |
| 28S-58 | CGCGCCGGCCITCACCITCATIGCGCCACIGCGGCITTCGIGCGAGCCCICGACICGCGCACGT | 87 |
| 28S-59 | GTIAGACICCTTGGICCGTGTTICAAGACGGGICGGGTGGGIAGCCGACGICGCCGCIGACCCC | 88 |
| 28S-60 | GTGCGCICGCICCGCCGICCCCCICTICGGGGGACGCGCGCGIGGCCCIGAGAGAACCICCCCC | 89 |
| 28S-61 | GGICCCGACGICGCGACCCGCICGGGGIGCACIGGGGACAGICCGCCCCGCCICCCGAICCGCG | 90 |
| 28S-62 | CGCIGCACCCICCCCGICGCCGGIGCGGGIGCGCGGGGAGGAIGGGIGGGAGAGCGGICGCGCC | 91 |
| 28S-63 | GIGGGAGGGGIGGCCCGGICCCCCCACGAGIAGACGCCGICGCGCCICCICGGGGIAGACCCCC | 92 |
| 28S-64 | CICGCGGGGGATICCCCGCGGGGGIGGGIGCCGGGAGGGGGIAGAGCGCGGIGACGGGICICGC | 93 |
| 28S-65 | TCCCICGGCCCCGGGATICGGCGAGIGCTGCIGCCGGGGGGGCIGIAACACICGGGGGGGGITT | 94 |
| 28S-66 | CGGICCCGCCGICGCCGCIGCCGCCGCIACCGCIGCCGCCGCCGCCGICCCGAICCICGCGCCC | 95 |
| 28S-67 | ICCCGAGGGAGGAIGCGGGGCCIGGGGICGIAGACGGGGGAGIAGGAGGACIGACGGAIGGACG | 96 |
| 28S-68 | GACGGIGCCCCCIGAGCCICCTICCCCGCCGGICCTICCCAGCCGICCCGGAGCCGGICGCGGC | 97 |
| 28S-69 | GCACIGCCGCGGIGGAAAIGCGCCCIGCGGCIGCCGGICGCCGGICGGGGGACGGICCCCCGCC | 98 |
| 28S-70 | GACICCACCCCCGGICCCGCICGCCCACICCCGCACCIGCCGGAGCICGCCCCCICCGGGIAGG | 99 |
| 28S-71 | AGGAIGAGGGGCIGCGGGGGAAIGGAGGGIGGGIGGAGGGGICGGGAGGAAIGGGIGGCGGGAA | 100 |
| 28S-72 | AGAICCGCCGGGICGCCGACACIGCCGGACCCGICGCCGGGITGAAICCICCGGGCGGACIGCG | 101 |
| 28S-73 | CGGAICCCACCCGITTACCTCTIAACGGTTICACGCCCICTTGAACICTCTCITCAAAGITCTT | 102 |
| 28S-74 | TTCAACITTCCCTIACGGTACTIGTTGACIATCGGICTCGIGCCGGTATITAGCCTIAGATGGA | 103 |
| 28S-75 | GTTIACCACCCGCITTGGGCIGCATICCCAAGCAICCCGACICCGGGAIGACCCGGGICCGGCG | 104 |
| 28S-76 | CGCCGIGGGCCGCIACCGGCCICACACCGICCACGGGCIGGGCCICGAICAGAAGGACITGGGC | 105 |
| 28S-77 | CCCCCACGAGIGGCGCCGGGIAGCGGGICTICCGIACGCCACATGICCCGCGCICCGCIGCGGG | 106 |
| 28S-78 | GCGGGGAITCGGCGCIGGGCTCTICCCTGITCACICGCCGTTACIGAGGGAAICCTGGITAGTT | 107 |
| 28S-79 | TCTICTCCTCCGCIGACTAATAIGCTTAAAITCAGCGGGICGCCACGICTGAICTGAGGICGCG | 108 |

I: T modified with biotin.

### Example: ssDNA Probes Hybridization→Streptavidin-Resins

1. Biotinylated ssDNA Probes Preparation

Sequences of the modified single strand DNA probes targeting the full length sequences of human 18S and human 28S rRNA are shown in Table 3. Wherein A means dA, T means dT, C means dC, G means dG, and I means amino-dT. DNA probes were synthesized by ABI DNA synthesizer with regular DMT-dN phosphoramidites and Amino-Modifier-C6-dT-CE phosphoramidite (Link Technologies Ltd., Scotland). After synthesis, modified DNA probes were treated with Sulfo-NHS-Biotin (ApexBio technology LLC, Houston, USA) for biotin labeling. In detail, DNA oligonucleotides were resolved in 0.1 M sodium bicarbonate to 200 μM. Sulfo-NHS-biotin was dissolved in 0.1 M sodium bicarbonate to 16 mM. Mix equal volume of probes and Sulfo-NHS-biotin solution and stay at room temperature overnight for labeling reaction. And then use desalt column to remove extra biotin.

DNA probes can also be directly synthesized by ABI DNA synthesizer with regular DMT-dN phosphoramidites and biotin-dT-CE Phosphoramidite. After synthesis, DNA probes would be biotin labeled probes. No further reaction as above mentioned is required.

The concentration of synthesized ssDNA probes were determined by spectrophotometer and adjusted to 1 μg/μL. The 18S probes were made by mixing 18S-1˜29 biotinylated ssDNA probes in the same molar ratio and adjust to a final concentration 400 ng/μL. The 28S probes were made by mixing 28S-1˜79 biotinylated ssDNA probes in the same molar ratio and adjust to a final concentration 1 μg/4.

2. Hybridization Biotinylated ssDNA Probes to Target RNA

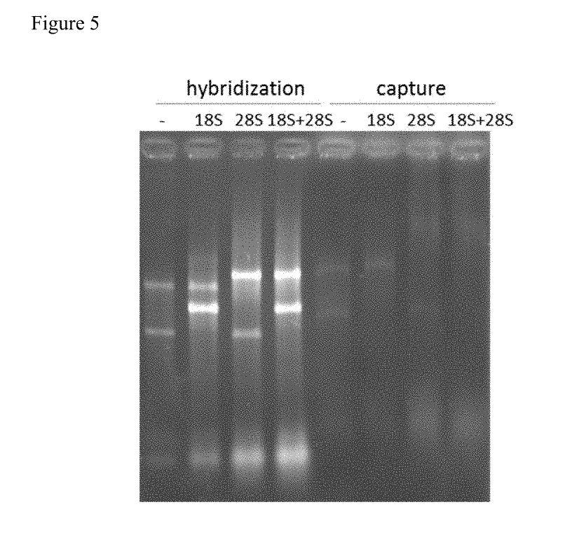

Targeted rRNA was mixed with 2× probes by weight. 1 μg RNA extracted from 293T cells was mixed with 400 ng 18S, 1.1 μg 28S, or 400 ng 18S+1.1 μg 28S biotinylated ssDNA probe mixture in 40 μl solution in a final concentration of 50 mM Tris-HCl, pH7.5, and 100 mM NaCl. To hybridize probes to target RNA, the mixture was heated to 70° C. for 5 min and then cool down to 25° C. for 5 min. Keep 5 μl hybridization products for gel loading (FIG. 5, lane 1-4, labeled hybridization).

3. Remove Biotin-ssDNA/RNA Hybrid by Streptavidin Coated Magnetic Beads (SMOBIO).

200 μl streptavidin coated magnetic beads were washed twice with DEPC-treated ddH₂O and once with 1× binding buffer (5 mM Tris-HCl pH7.5, 0.5 mM EDTA, 1M NaCl, 0.05% Tween20). After wash, streptavidin coated magnetic beads were added with 40 μl 2× binding buffer, RI (SMOBIO), and 30 μl hybridization products (from step 2) to capture biotin-ssDNA/RNA hybrid. Keep the mixture swirling at room temperature for 30 min and then 50° C. for 5 min. After removal of streptavidin coated magnetic beads/biotin-ssDNA/RNA hybrid, the residual solution revealed depletion efficiency by gel electrophoresis. (FIG. 5. lane 5-8, labeled capture).

While the present invention has been described with reference to what is considered to be specific embodiments, it is to be understood that the invention is not so limited. To the contrary, the invention is intended to cover various modifications and equivalents included within the spirit and scope of the appended claims.

What is claimed is:

1. A method of depleting target RNA from a nucleic acid sample comprising target and non-target RNA molecules, comprising:
(a) contacting the nucleic acid sample with at least one double strand DNA probe in a mixture, wherein each strand of the at least one double strand DNA probe is complementary to part of the target RNA and capable of specifically hybridizing to entire full length sequence of the target RNA; and
(b) contacting the mixture with RNase that specifically recognizes the DNA-RNA hybrid and degrades the target RNA in the DNA-RNA hybrid.

2. The method of claim 1, further comprises contacting the mixture with DNase to degrade residual DNA from the DNA-RNA hybrid after step (b).

3. The method of claim 1, wherein the target RNA is ribosomal RNA or transfer RNA.

4. The method of claim 1, wherein the nucleic acid sample comprise RNA extracted, isolated, or purified from a source selected from the group consisting of: a tissue sample, a cell sample, a paraffin-embedded sample, a paraffin-embedded formalin-fixed (FFPE) sample, and an environmental sample consisting of soil, water, growth medium, or a biological fluid or specimen.

5. The method of claim 1, wherein the RNase is RNase H.

6. The method of claim 2, wherein the DNase is DNase I.

7. A method of depleting or isolating target RNA from a nucleic acid sample comprising target and non-target RNA molecules, comprising:
(a) contacting the nucleic acid sample with at least one modified double strand DNA probe in a mixture, wherein the at least one modified double strand DNA probe is having at least one nucleotide modified with affinitive moiety or reactive moiety, wherein each strand of the at least one double strand DNA probe is complementary to part of the target RNA and capable of specifically hybridizing to the target RNA; and
(b) contacting the mixture with a matrix that specifically interacts with the at least one modified double strand DNA probe on the modified DNA-RNA hybrid, such that the modified DNA-RNA hybrid bind to the matrix and are removed from the mixture,
wherein the at least one modified double strand DNA probe is having at least one nucleotide modified with affinitive moiety and the matrix is affinitive matrix, or the at least one modified double strand DNA probe is having at least one nucleotide modified with reactive moiety and the matrix is reactive matrix.

8. The method of claim 7, wherein the at least one modified double strand DNA probe is biotinylated double strand DNA probe and the affinitive matrix is avidin matrix or streptavidin matrix.

9. The method of claim 8, wherein the biotinylated single strand DNA probes are prepared from reacting the multiplicity of modified single strand DNA probes are having at least one nucleotide modified with a first reactive moiety with a biotin modified with a second reactive moiety.

10. The method of claim 9, wherein the first reactive moiety is primary amine group and the second reactive moiety is N-hydroxysuccinimide group.

11. The method of claim 8, wherein the affinitive matrix is prepared from reacting a streptavidin which is modified with a first reactive moiety with a matrix having a second reactive moiety.

12. The method of claim 9, wherein the first reactive moiety is primary amine group and the second reactive moiety is N-hydroxysuccinimide group.

13. The method of claim 7, wherein the reactive moiety is alkyne group and the reactive matrix is containing azide group, the reactive moiety is azide group and the reactive matrix is containing alkyne group, the reactive moiety is thioester group and the reactive matrix is containing N-terminal cysteine group, the reactive moiety is N-terminal cysteine group and the reactive matrix is containing thioester group, the reactive moiety is primary amine group and the reactive matrix is containing N-hydroxysuccinimide group, or the reactive moiety is N-hydroxysuccinimide group and the reactive matrix is containing primary amine group.

14. The method of claim 7, wherein the target RNA is ribosomal RNA or transfer RNA.

15. The method of claim 7, wherein the nucleic acid sample comprise RNA extracted, isolated, or purified from a source selected from the group consisting of: a tissue sample, a cell sample, a paraffin-embedded sample, a paraffin-embedded formalin-fixed (FFPE) sample, and an environmental sample consisting of soil, water, growth medium, or a biological fluid or specimen.

16. The method of claim 7, wherein the matrix is selected from the group consisting of microtitre plate, magnetic bead, non-magnetic bead, sedimentation particle, and affinity chromatography column.

17. A method of preparing a denatured double strand DNA in a nucleic acid sample for hybridization, comprising:
(a) contacting the nucleic acid sample for hybridization with double strand DNA in a hybridization buffer; and
(b) heating the mixture to a temperature from 68 to 90° C. to obtain the denatured double strand DNA,
wherein the hybridization buffer comprises formamide in a concentration from 40% to 70% by volume.

18. The method of claim 17, wherein the temperature is 70° C.

19. The method of claim 17, wherein the formamide is in a concentration of 40% by volume.


SEQUENCE LISTING (144 sequences)

SEQ ID NO: 1 - DNA, 64 bp, Artificial Sequence
  primer
  misc_feature (1)..(64)
taatgatccttccgcaggttcacctacggaaaccttgttacgacttttacttcctctagatagt

SEQ ID NO: 2 - DNA, 64 bp, Artificial Sequence
  primer
  misc_feature (1)..(64)
aagttcgaccgtcttctcagcgctccgccagggccgtgggccgaccctggcggggccgatccga

SEQ ID NO: 3 - DNA, 64 bp, Artificial Sequence
  primer
  misc_feature (1)..(64)
ggcctcactaaaccatccaatcggtagtagcgacgggcggtgtgtacaaaggtcagggacttaa

SEQ ID NO: 4 - DNA, 64 bp, Artificial Sequence
  primer
  misc_feature (1)..(64)
tcaacgcaagcttatgacccgcacttactgggaattcctcgttcatggggaataattgcaatcc

SEQ ID NO: 5 - DNA, 64 bp, Artificial Sequence
  primer
  misc_feature (1)..(64)
cgatccccatcacgaatggggttcaacgggttacccgcgcctgccggcgtagggtaggcacacg

SEQ ID NO: 6 - DNA, 64 bp, Artificial Sequence
  primer
  misc_feature (1)..(64)
tgagccagtcagtgtagcgcgcgtgcagccccggacatctaagggcatcacagacctgttattg

SEQ ID NO: 7 - DNA, 64 bp, Artificial Sequence
  primer
  misc_feature (1)..(64)
tcaatctcgggtggctgaacgccacttgtccctctaagaagttgggggacgccgaccgctcggg

SEQ ID NO: 8 - DNA, 64 bp, Artificial Sequence
  primer
  misc_feature (1)..(64)
gtcgcgtaactagttagcatgccagagtctcgttcgttatcggaattaaccagacaaatcgctc

SEQ ID NO: 9 - DNA, 64 bp, Artificial Sequence
  primer
  misc_feature (1)..(64)
accaactaagatcggccatgcaccaccatccacggaatcgagaaagagctatcaatctgtcaat

SEQ ID NO: 10 - DNA, 64 bp, Artificial Sequence
  primer
  misc_feature (1)..(64)
ctgtccgtgtccgggccgggtgaggtttcccgtgttgagtcaaattaagccgcaggctccactc

SEQ ID NO: 11 - DNA, 64 bp, Artificial Sequence
  primer
  misc_feature (1)..(64)
tggtggtgcccttccgtcaattcctttaagtttcagctttgcaaccatactccccctggaaccc

SEQ ID NO: 12 - DNA, 64 bp, Artificial Sequence
  primer
  misc_feature (1)..(64)
aagactttggtttcccggaagctgcccggcgggtcatgggaataacgccgccgcatcgccggtc

SEQ ID NO: 13 - DNA, 64 bp, Artificial Sequence
  primer
  misc_feature (1)..(64)
gcatcgtttatggtcggaactacgacggtatctgatcgtcttcgaacctccgactttcgttctt

SEQ ID NO: 14 - DNA, 64 bp, Artificial Sequence
  primer
  misc_feature (1)..(64)
attaatgaaaacattcttggcaaatgctttcgctctggtccgtcttgcgccggtccaagaattt

SEQ ID NO: 15 - DNA, 64 bp, Artificial Sequence
  primer
  misc_feature (1)..(64)
acctctagcggcgcaatacgaatgcccccggccgtccctcttaatcatggcctcagttccgaaa

SEQ ID NO: 16 - DNA, 62 bp, Artificial Sequence
  primer
  misc_feature (1)..(62)
accaacaaaatagaaccgcggtcctattccattattcctagctgcggtatccaggcggctcg

SEQ ID NO: 17 - DNA, 64 bp, Artificial Sequence
  primer
  misc_feature (1)..(64)
ggcctgctttgaacactctaattttttcaaagtaaacgcttcgggcctcgcgggacactcagct

SEQ ID NO: 18 - DNA, 64 bp, Artificial Sequence
  primer
  misc_feature (1)..(64)
aagagcatcgaggggtcgccgagagtcaagggtcggggactggcggtggctcgcctcgcggcgg

SEQ ID NO: 19 - DNA, 64 bp, Artificial Sequence
  primer
  misc_feature (1)..(64)
accgtccgcccgctcccaagatccaactacgagctttttaactgcagcaactttaatatacgct

SEQ ID NO: 20 - DNA, 64 bp, Artificial Sequence
  primer
  misc_feature (1)..(64)
attggagctggaattaccgcggctgctggcaccagacttgccctccaatggatcctcgttaaag

SEQ ID NO: 21 - DNA, 64 bp, Artificial Sequence
  primer
  misc_feature (1)..(64)
gatttaaagtggactcattccaattacagggcctcgaaagagtcctgtattgttatttttcgtc

SEQ ID NO: 22 - DNA, 64 bp, Artificial Sequence
  primer
  misc_feature (1)..(64)
actacctccccgggtcgggagtgggtaatttgcgcgcctgctgccttccttggatgtggtagcc

SEQ ID NO: 23 - DNA, 64 bp, Artificial Sequence
  primer
  misc_feature (1)..(64)
gtttctcaggctccctctccggaatcgaaccctgattccccgtcacccgtggtcaccatggtag

SEQ ID NO: 24 - DNA, 64 bp, Artificial Sequence
  primer
  misc_feature (1)..(64)
gcacggcgactaccatcgaaagttgatagggcagacgttcgaatgggtcgtcgccgccactggg

SEQ ID NO: 25 - DNA, 64 bp, Artificial Sequence
  primer
  misc_feature (1)..(64)
gcgtgcgatcggcccgaggttatctagagtcaccaaagccgtcggcgcccgtcccccggcctgg

SEQ ID NO: 26 - DNA, 64 bp, Artificial Sequence
  primer
  misc_feature (1)..(64)
cctgagaggggctgaccgggttggttttgatctgataaatgcacgcatcccccccgtgaagggg

SEQ ID NO: 27 - DNA, 64 bp, Artificial Sequence
  primer
  misc_feature (1)..(64)
tcagcgcccgtcggcatgtattagctctagaattaccacagttatccaagtaggagaggagtga

SEQ ID NO: 28 - DNA, 64 bp, Artificial Sequence
  primer
  misc_feature (1)..(64)
gcgatcaaaggaaccataactgatttaatgagccattcgcagtttcactgtaccggccgtgcgt

SEQ ID NO: 29 - DNA, 63 bp, Artificial Sequence
  primer
  misc_feature (1)..(63)
actcagacatgcatggcttaatctttgagacaagcatatgctactggcaggatcaaccaggta

SEQ ID NO: 30 - DNA, 64 bp, Artificial Sequence
  primer
  misc_feature (1)..(64)
gacaaacccttgtgtcgagggctgactttcaatagatcgcagcgagggagctgctctgctacgt

SEQ ID NO: 31 - DNA, 64 bp, Artificial Sequence
  primer
  misc_feature (1)..(64)
acgtaacccctacccagtagcaggtcgtctacgaatggtttagcgccaggttccccacgaacgt

SEQ ID NO: 32 - DNA, 64 bp, Artificial Sequence
  primer
  misc_feature (1)..(64)
gcggtgcgtgacgggcgaggtggcggccgcctctccggccgtgccccgtttcccaggatgaagg

SEQ ID NO: 33 - DNA, 64 bp, Artificial Sequence
  primer
  misc_feature (1)..(64)
gcactccgcacctgaccccggtcccggcgctcggcggggtacgcgccctcccgtgctcgcgggg

SEQ ID NO: 34 - DNA, 64 bp, Artificial Sequence
  primer
  misc_feature (1)..(64)
cgcgtggaggtggggggcggcctgccggcgggtacaggcggtggaccggctatccgagtccaac

SEQ ID NO: 35 - DNA, 64 bp, Artificial Sequence
  primer
  misc_feature (1)..(64)
gaggctccgcggcgctgccgtatcgttccgcctgggcgggattctgacttagaggcgttcagtc

SEQ ID NO: 36 - DNA, 64 bp, Artificial Sequence
  primer
  misc_feature (1)..(64)
ataatcccacagatggtagcttcgccccattggctcctcagccaagcacatacaccaaatgtct

SEQ ID NO: 37 - DNA, 64 bp, Artificial Sequence
  primer
  misc_feature (1)..(64)
gaacctgcggttcctctcgtactgagcaggattaccatggcaacaacacatcatcagtagggta

SEQ ID NO: 38 - DNA, 64 bp, Artificial Sequence
  primer
  misc_feature (1)..(64)
aaactaacctgtctcacgacggtctaaacccagctcacgttccctattagtgggtgaacaatcc

SEQ ID NO: 39 - DNA, 64 bp, Artificial Sequence
  primer
  misc_feature (1)..(64)
acgcttggtgaattctgcttcacaatgataggaagtgccgacatcgaaggatcaaaatgcgacg

SEQ ID NO: 40 - DNA, 64 bp, Artificial Sequence
  primer
  misc_feature (1)..(64)
tcgctatgaacgcttggccgccacaagccagttatccctgtggtaacttttctgacacctcctg

SEQ ID NO: 41 - DNA, 64 bp, Artificial Sequence
  primer
  misc_feature (1)..(64)
cttaaaacccaaaaggtcagaaggatcgtgaggccccgctttcacggtctgtattcgtactgaa

SEQ ID NO: 42 - DNA, 64 bp, Artificial Sequence
  primer
  misc_feature (1)..(64)
aatcaagatcaagcgagcttttgcccttctgctccacgggaggtttctgtcctccctgagctcg

SEQ ID NO: 43 - DNA, 64 bp, Artificial Sequence
  primer
  misc_feature (1)..(64)
ccttaggacacctgcgttaccgtttgacaggtgtaccgccccagtcaaactccccacctggcac

SEQ ID NO: 44 - DNA, 64 bp, Artificial Sequence
  primer
  misc_feature (1)..(64)
tgtccccggagcgggtcgcgcctggccgtcgcgcggccgtgcgcttggcgccagaagtgagagc

SEQ ID NO: 45 - DNA, 64 bp, Artificial Sequence
  primer
  misc_feature (1)..(64)
cctcggggctcgcccccccgcctcaccgggtcagtgaaaaaacgatcagagtagtggtatttca

SEQ ID NO: 46 - DNA, 64 bp, Artificial Sequence
  primer
  misc_feature (1)..(64)
cggctgcccgctgggtcggcggacctcgcctcgggcccctcgcggggacatcggtggggcgccg

SEQ ID NO: 47 - DNA, 64 bp, Artificial Sequence
  primer
  misc_feature (1)..(64)
gggcctcccacttattctacacctctcatgtctcttcaccgtgccagactagagtcaagctcaa

SEQ ID NO: 48 - DNA, 64 bp, Artificial Sequence
  primer
  misc_feature (1)..(64)
cagggtcttctttccccgctgattccgccaagcccgttcccttggctgtggtttcgctggatag

SEQ ID NO: 49 - DNA, 64 bp, Artificial Sequence
  primer
  misc_feature (1)..(64)
taggtagggacagtgggaatctcgttcatccattcatgcgcgtcactaattagatgacgaggca

SEQ ID NO: 50 - DNA, 64 bp, Artificial Sequence
  primer
  misc_feature (1)..(64)
tttggctaccttaagagagtcatagttactcccgccgtttacccgcgcttcattgaatttcttc

SEQ ID NO: 51 - DNA, 64 bp, Artificial Sequence
  primer
  misc_feature (1)..(64)
actttgacattcagagcactgggcagaaatcacatcgcgtcaacacccgctgcgggccttcgcg

SEQ ID NO: 52 - DNA, 64 bp, Artificial Sequence
  primer
  misc_feature (1)..(64)
atgctttgttttaattaaacagtcggattcccctggtccgcaccagttctaagtcggctgctag

SEQ ID NO: 53 - DNA, 64 bp, Artificial Sequence
  primer
  misc_feature (1)..(64)
cgccgtccgagtcgaggtgccgcgctgaaccgtggccctgggggcggacccgtcggtggggacc

SEQ ID NO: 54 - DNA, 64 bp, Artificial Sequence
  primer
  misc_feature (1)..(64)
cccgtggcccctccgccgcctgccgccgtcgccgccgtgcgccgtggaggatggtggaacgggg

SEQ ID NO: 55 - DNA, 64 bp, Artificial Sequence
  primer
  misc_feature (1)..(64)
gcgtacggggtcgggggggtagggcgggggtacgaaccgtcccgctccgccgtccgctgaccgc

SEQ ID NO: 56 - DNA, 64 bp, Artificial Sequence
  primer
  misc_feature (1)..(64)
gccgtccgaccgctccccgcccctagcggactcgcgcgctacgagacgtggggtgggggtgggg

SEQ ID NO: 57 - DNA, 64 bp, Artificial Sequence
  primer
  misc_feature (1)..(64)
gctcgccgtcgcccgctgggctccccgggggcgtccgcgacgcctgccgcagctggggcgatcc

SEQ ID NO: 58 - DNA, 64 bp, Artificial Sequence
  primer
  misc_feature (1)..(64)
acgggaagtgcccggctcgcgtccagagtcgccgctgccgccggctccccgggtgccctggccc

SEQ ID NO: 59 - DNA, 64 bp, Artificial Sequence
  primer
  misc_feature (1)..(64)
ccctcgcgtgggaccgtgcccctgccgccggggcctcgcggcgggctgctgccggcccctgccg

SEQ ID NO: 60 - DNA, 64 bp, Artificial Sequence
  primer
  misc_feature (1)..(64)
cccctacccttctccccccgccgccgtccccacgcggtgctcccccggggatggggtaggacgg

SEQ ID NO: 61 - DNA, 64 bp, Artificial Sequence
  primer
  misc_feature (1)..(64)
agcggtggagagagatagagatagggctcggtgcggggaggtagcgagcggcgtgcgcggggtg

SEQ ID NO: 62 - DNA, 64 bp, Artificial Sequence
  primer
  misc_feature (1)..(64)
ggtcgggggagggtcgcgagtggggtgccccgggcgtggggggggcgtcggcgcctcgtccagc

SEQ ID NO: 63 - DNA, 64 bp, Artificial Sequence
  primer
  misc_feature (1)..(64)
gtggtgcgcgcccatccccgcttcgcgccctagcccgaccgatccagcccttagagccaatcct

SEQ ID NO: 64 - DNA, 64 bp, Artificial Sequence
  primer
  misc_feature (1)..(64)
tatcccgaagttacggatccggcttgccgacttcccttacctacattgttccaacatgccagag

SEQ ID NO: 65 - DNA, 64 bp, Artificial Sequence
  primer
  misc_feature (1)..(64)
gctgttcaccttggagacctgctgcggatatgggtacggcccggcgcgagatttacaccctctc

SEQ ID NO: 66 - DNA, 64 bp, Artificial Sequence
  primer
  misc_feature (1)..(64)
ccccggattttcaagggctagcgagagctcaccggatgccgccggatccgcgacgctttccaag

SEQ ID NO: 67 - DNA, 64 bp, Artificial Sequence
  primer
  misc_feature (1)..(64)
gcacgggcccctctctcggggtgaacccattccagggtgccctgcccttcacaaagtaaagaga

SEQ ID NO: 68 - DNA, 64 bp, Artificial Sequence
  primer
  misc_feature (1)..(64)
actctccccggggctcccgccggcttctccgggatcggtcgcgttaccgcactggacgcctcgc

SEQ ID NO: 69 - DNA, 64 bp, Artificial Sequence
  primer
  misc_feature (1)..(64)
ggcgcccatctccgccactccggattcggggatctgaacccgactccctttcgatcggccgagg

SEQ ID NO: 70 - DNA, 64 bp, Artificial Sequence
  primer
  misc_feature (1)..(64)
caacgtaggccatcgcccgtcccttcggaacggcgctcgcccatctctcaggaccgactgaccc

SEQ ID NO: 71 - DNA, 64 bp, Artificial Sequence
  primer
  misc_feature (1)..(64)
atgttcaactgctgttcacatggaacccttctccacttcggccttcaaagttctcgtttgaata

SEQ ID NO: 72 - DNA, 64 bp, Artificial Sequence
  primer
  misc_feature (1)..(64)
tttgctactaccaccaagatctgcacctgcggcggctccacccgggcccgcgccctaggcttca

SEQ ID NO: 73 - DNA, 64 bp, Artificial Sequence
  primer
  misc_feature (1)..(64)
aggctcaccgcagcggccctcctactcgtcgcggcgtagcgtccgcggggctccgggtgcgggg

SEQ ID NO: 74 - DNA, 64 bp, Artificial Sequence
  primer
  misc_feature (1)..(64)
agctgggcgtgggcggtaggagggtaggaggcgtgggggggtgggcgggggaatgatcccacac

SEQ ID NO: 75 - DNA, 64 bp, Artificial Sequence
  primer
  misc_feature (1)..(64)
ccccgtcgccgccgctgcctccgccctccgacgtacaccacatgcgcgcgctcgctcgccgccc

SEQ ID NO: 76 - DNA, 64 bp, Artificial Sequence
  primer
  misc_feature (1)..(64)
ccgccgctcccgtccactctcgactgccggcgatggccgggtatgggcccgacgctccagcgcc

SEQ ID NO: 77 - DNA, 64 bp, Artificial Sequence
  primer
  misc_feature (1)..(64)
atccattttcagggctagttgattcggcaggtgagttgttacacactccttagcggattccgac

SEQ ID NO: 78 - DNA, 64 bp, Artificial Sequence
  primer
  misc_feature (1)..(64)
ttccatggccaccgtcctgctgtctatatcaaccaacaccttttctggggtctgatgagcgtcg

SEQ ID NO: 79 - DNA, 64 bp, Artificial Sequence
  primer
  misc_feature (1)..(64)
gcatcgggcgccttaacccggcgttcggttcatcccgcagcgccagttctgcttaccaaaagtg

SEQ ID NO: 80 - DNA, 64 bp, Artificial Sequence
  primer
  misc_feature (1)..(64)
gcccactaggcactcgcattccacgcccggctccacgccagtgagccgggcttcttacccattt

SEQ ID NO: 81 - DNA, 64 bp, Artificial Sequence
  primer
  misc_feature (1)..(64)
aaagtttgagaataggttgagatcgtttcggccccaagacctctaatcattcgctttaccggat

SEQ ID NO: 82 - DNA, 64 bp, Artificial Sequence
  primer
  misc_feature (1)..(64)
aaaactgcgtggcgggggtgcgtcgggtctgcgagagcgccagctatcctgagggaaacttcgg

SEQ ID NO: 83 - DNA, 64 bp, Artificial Sequence
  primer
  misc_feature (1)..(64)
agggaaccagctactagatggttcgattagtctttcgcccctatacccaggtcggatgaccgat

SEQ ID NO: 84 - DNA, 64 bp, Artificial Sequence
  primer
  misc_feature (1)..(64)
ttgcacgtcaggaccgctacggacctccaccagagtttcctctggcttcgccctgcccaggcat

SEQ ID NO: 85 - DNA, 64 bp, Artificial Sequence
  primer
  misc_feature (1)..(64)
agttcaccatctttcgggtcctaacacgtgcgctcgtgctccacctccccggcgcggctggcga

SEQ ID NO: 86 - DNA, 64 bp, Artificial Sequence
  primer
  misc_feature (1)..(64)
gacgggccggtggtgcgccctcggcggactggagaggcctcgggatcccacctcggcctgcgag

SEQ ID NO: 87 - DNA, 64 bp, Artificial Sequence
  primer
  misc_feature (1)..(64)
cgcgccggccttcaccttcattgcgccactgcggctttcgtgcgagccctcgactcgcgcacgt

SEQ ID NO: 88 - DNA, 64 bp, Artificial Sequence
  primer
  misc_feature (1)..(64)
gttagactccttggtccgtgtttcaagacgggtcgggtgggtagccgacgtcgccgctgacccc

SEQ ID NO: 89 - DNA, 64 bp, Artificial Sequence
  primer
  misc_feature (1)..(64)
gtgcgctcgctccgccgtccccctcttcgggggacgcgcgcgtggccctgagagaacctccccc

SEQ ID NO: 90 - DNA, 64 bp, Artificial Sequence
  primer
  misc_feature (1)..(64)
ggtcccgacgtcgcgacccgctcggggtgcactggggacagtccgccccgcctcccgatccgcg

SEQ ID NO: 91 - DNA, 64 bp, Artificial Sequence
  primer
  misc_feature (1)..(64)
cgctgcaccctccccgtcgccggtgcgggtgcgcggggaggatgggtgggagagcggtcgcgcc

SEQ ID NO: 92 - DNA, 64 bp, Artificial Sequence
  primer
  misc_feature (1)..(64)
gtgggaggggtggcccggtccccccacgagtagacgccgtcgcgcctcctcggggtagaccccc

SEQ ID NO: 93 - DNA, 64 bp, Artificial Sequence
  primer
  misc_feature (1)..(64)
ctcgcgggggattccccgcgggggtgggtgccgggagggggtagagcgcggtgacgggtctcgc

SEQ ID NO: 94 - DNA, 64 bp, Artificial Sequence
  primer
  misc_feature (1)..(64)
tccctcggccccgggattcggcgagtgctgctgccgggggggctgtaacactcggggggggttt

SEQ ID NO: 95 - DNA, 64 bp, Artificial Sequence
  primer
  misc_feature (1)..(64)
cggtcccgccgtcgccgctgccgccgctaccgctgccgccgccgccgtcccgatcctcgcgccc

SEQ ID NO: 96 - DNA, 64 bp, Artificial Sequence
  primer
  misc_feature (1)..(64)
tcccgagggaggatgcggggcctggggtcgtagacgggggagtaggaggactgacggatggacg

SEQ ID NO: 97 - DNA, 64 bp, Artificial Sequence
  primer
  misc_feature (1)..(64)
gacggtgccccctgagcctccttccccgccggtccttcccagccgtcccggagccggtcgcggc

SEQ ID NO: 98 - DNA, 64 bp, Artificial Sequence
  primer
  misc_feature (1)..(64)
gcactgccgcggtggaaatgcgccctgcggctgccggtcgccggtcgggggacggtcccccgcc

SEQ ID NO: 99 - DNA, 64 bp, Artificial Sequence
  primer
  misc_feature (1)..(64)
gactccacccccggtcccgctcgcccactcccgcacctgccggagctcgccccctccgggtagg

SEQ ID NO: 100 - DNA, 64 bp, Artificial Sequence
  primer
  misc_feature (1)..(64)
aggatgaggggctgcgggggaatggagggtgggtggaggggtcgggaggaatgggtggcgggaa

SEQ ID NO: 101 - DNA, 64 bp, Artificial Sequence
  primer
  misc_feature (1)..(64)
agatccgccgggtcgccgacactgccggacccgtcgccgggttgaatcctccgggcggactgcg

SEQ ID NO: 102 - DNA, 64 bp, Artificial Sequence
  primer
  misc_feature (1)..(64)
cggatcccacccgtttacctcttaacggtttcacgccctcttgaactctctcttcaaagttctt

SEQ ID NO: 103 - DNA, 64 bp, Artificial Sequence
  primer
  misc_feature (1)..(64)
ttcaactttcccttacggtacttgttgactatcggtctcgtgccggtatttagccttagatgga

SEQ ID NO: 104 - DNA, 64 bp, Artificial Sequence
  primer
  misc_feature (1)..(64)
gtttaccacccgctttgggctgcattcccaagcatcccgactccgggatgacccgggtccggcg

SEQ ID NO: 105 - DNA, 64 bp, Artificial Sequence
  primer
  misc_feature (1)..(64)
cgccgtgggccgctaccggcctcacaccgtccacgggctgggcctcgatcagaaggacttgggc

SEQ ID NO: 106 - DNA, 64 bp, Artificial Sequence
  primer
  misc_feature (1)..(64)
cccccacgagtggcgccgggtagcgggtcttccgtacgccacatgtcccgcgctccgctgcggg

SEQ ID NO: 107 - DNA, 64 bp, Artificial Sequence
  primer
  misc_feature (1)..(64)
gcggggattcggcgctgggctcttccctgttcactcgccgttactgagggaatcctggttagtt

SEQ ID NO: 108 - DNA, 64 bp, Artificial Sequence
  primer
  misc_feature (1)..(64)
tcttctcctccgctgactaatatgcttaaattcagcgggtcgccacgtctgatctgaggtcgcg

SEQ ID NO: 109 - DNA, 28 bp, Artificial Sequence
  primer
  misc_feature (1)..(28)
cagtaaggaggtgatccaaccgcaggtt

SEQ ID NO: 110 - DNA, 30 bp, Artificial Sequence
  primer
  misc_feature (1)..(30)
ccaacatttcacaacacgagctgacgacag

SEQ ID NO: 111 - DNA, 27 bp, Artificial Sequence
  primer
  misc_feature (1)..(27)
ctctacgcatttcaccgctacacctgg

SEQ ID NO: 112 - DNA, 29 bp, Artificial Sequence
  primer
  misc_feature (1)..(29)
cccgtaggagtctggaccgtgtctcagtt

SEQ ID NO: 113 - DNA, 29 bp, Artificial Sequence
  primer
  misc_feature (1)..(29)
cagaaggttaagcctcacggttcattagt

SEQ ID NO: 114 - DNA, 29 bp, Artificial Sequence
  primer
  misc_feature (1)..(29)
cccaggatgtgatgagccgacatcgaggt

SEQ ID NO: 115 - DNA, 29 bp, Artificial Sequence
  primer
  misc_feature (1)..(29)
ccatgcagactggcgtccacacttcaaag

SEQ ID NO: 116 - DNA, 30 bp, Artificial Sequence
  primer
  misc_feature (1)..(30)
ccactttcgtgtttgcacagtgctgtgttt

SEQ ID NO: 117 - DNA, 30 bp, Artificial Sequence
  primer
  misc_feature (1)..(30)
ccttcgcagtaacaccaagtacaggaatat

SEQ ID NO: 118 - DNA, 30 bp, Artificial Sequence
  primer
  misc_feature (1)..(30)
cccacatcgtttcccacttaaccatgactt

SEQ ID NO: 119 - DNA, 29 bp, Artificial Sequence
  primer
  misc_feature (1)..(29)
cccagttaagactcggtttcccttcggct

SEQ ID NO: 120 - DNA, 29 bp, Artificial Sequence
  primer
  misc_feature (1)..(29)
ccctgtatcgcacgcctttccagacgctt

SEQ ID NO: 121 - DNA, 28 bp, Artificial Sequence
  primer
  misc_feature (1)..(28)
cagtaaggaggtgatccaaccgcaggtt

SEQ ID NO: 122 - DNA, 22 bp, Artificial Sequence
  primer
  misc_feature (1)..(22)
gttaagtcccgcaacgagcgca

SEQ ID NO: 123 - DNA, 30 bp, Artificial Sequence; identical to SEQ ID NO: 110
  primer
  misc_feature (1)..(30)

SEQ ID NO: 124 - DNA, 22 bp, Artificial Sequence
  primer
  misc_feature (1)..(22)
atctggaggaataccggtggcg

SEQ ID NO: 125 - DNA, 27 bp, Artificial Sequence
  primer
  misc_feature (1)..(27)
ctctacgcatttcaccgctacacctgg

SEQ ID NO: 126 - DNA, 23 bp, Artificial Sequence
  primer
  misc_feature (1)..(23)
aggcagcagtggggaatattgca

SEQ ID NO: 127 - DNA, 29 bp, Artificial Sequence
  primer
  misc_feature (1)..(29)
cccgtaggagtctggaccgtgtctcagtt

SEQ ID NO: 128 - DNA, 30 bp, Artificial Sequence
  primer
  misc_feature (1)..(30)
gcggatccaaattgaagagtttgatcatgg

SEQ ID NO: 129 - DNA, 29 bp, Artificial Sequence
  primer
  misc_feature (1)..(29)
cagaaggttaagcctcacggttcattagt

SEQ ID NO: 130 - DNA, 22 bp, Artificial Sequence
  primer
  misc_feature (1)..(22)
gctgaagtaggtcccaagggta

SEQ ID NO: 131 - DNA, 29 bp, Artificial Sequence
  primer
  misc_feature (1)..(29)
cccaggatgtgatgagccgacatcgaggt

SEQ ID NO: 132 - DNA, 22 bp, Artificial Sequence
  primer
  misc_feature (1)..(22)
agccgaccttgaaataccaccc

SEQ ID NO: 133 - DNA, 29 bp, Artificial Sequence
  primer
  misc_feature (1)..(29)
ccatgcagactggcgtccacacttcaaag

SEQ ID NO: 134 - DNA, 22 bp, Artificial Sequence
  primer
  misc_feature (1)..(22)
acgtatacggtgtgacgcctgc

SEQ ID NO: 135 - DNA, 30 bp, Artificial Sequence; identical to SEQ ID NO: 116
  primer
  misc_feature (1)..(30)

SEQ ID NO: 136 - DNA, 22 bp, Artificial Sequence
  primer
  misc_feature (1)..(22)
ggggacggagaaggctatgttg

SEQ ID NO: 137 - DNA, 30 bp, Artificial Sequence; identical to SEQ ID NO: 117
  primer
  misc_feature (1)..(30)

SEQ ID NO: 138 - DNA, 22 bp, Artificial Sequence
  primer
  misc_feature (1)..(22)
aaggcccagacagccaggatgt

SEQ ID NO: 139 - DNA, 30 bp, Artificial Sequence; identical to SEQ ID NO: 118
  primer
  misc_feature (1)..(30)

SEQ ID NO: 140 - DNA, 22 bp, Artificial Sequence
  primer
  misc_feature (1)..(22)
cgttaagttgcagggtatagac

SEQ ID NO: 141 - DNA, 29 bp, Artificial Sequence
  primer
  misc_feature (1)..(29)
cccagttaagactcggtttcccttcggct

SEQ ID NO: 142 - DNA, 22 bp, Artificial Sequence
  primer
  misc_feature (1)..(22)
tgacagccccgtacacaaaaat

SEQ ID NO: 143 - DNA, 29 bp, Artificial Sequence
  primer
  misc_feature (1)..(29)
ccctgtatcgcacgcctttccagacgctt

SEQ ID NO: 144 - DNA, 30 bp, Artificial Sequence
  primer
  misc_feature (1)..(30)
aaggatccggttaagcgactaagcgtacac